(19) United States
(12) Patent Application Publication
(10) Pub. No.: US 2025/0339675 A1
(43) Pub. Date: Nov. 6, 2025

(54) WEARABLE HEADBAND DEVICE FOR NON-INVASIVE BRAIN STIMULATION

(71) Applicant: NEURACLE HEALTH PRIVATE LIMITED, New Delhi (IN)

(72) Inventors: Lakshay Sahni, New Delhi (IN); Swapniil Kanwar, New Delhi (IN); Ramya Yellparagada, New Delhi (IN)

(73) Assignee: NEURACLE HEALTH PRIVATE LIMITED, New Delhi (IN)

(21) Appl. No.: 19/198,176

(22) Filed: May 5, 2025

(30) Foreign Application Priority Data
May 3, 2024 (IN) 202411035193

(51) Int. Cl. A61N 1/04; A61N 1/02; A61N 1/08; A61N 1/36

(52) U.S. Cl. CPC A61N 1/0484; A61N 1/025; A61N 1/0456; A61N 1/08; A61N 1/3603

(57) ABSTRACT

A wearable headband device for non-invasive brain stimulation includes a first stimulation electrode (102A), a second stimulation electrode (102B) and an electronic circuit (106) for switching a direction of flow of current between the first stimulation electrode (102A) and the second stimulation electrode (102B). The electronic circuit (106) includes a first circuit (116) including a pair of first type of switches (118) and a pair of second type of switches (120). Furthermore, a protection circuit (124) is configured to detect and control the flow of current passing through the first stimulation electrode (102A) and the second stimulation 10 electrode (102B). Further, a microcontroller (126) is configured to control the pair of first type of switches (118) and the pair of second type of switches (120) of the first circuit (116) for the switching the direction of flow of current between the first stimulation electrode (102A) and the second stimulation electrode (102B).

### CROSS-REFERENCE TO RELATED APPLICATIONS/INCORPORATION BY REFERENCE

This Application makes reference to, claims priority to, and claims benefit from Indian Non-Provisional application Ser. No. 202411035193 filed on May 3, 2024. The above-referenced Applications are hereby incorporated herein by reference in their entirety.

### TECHNICAL FIELD

The present disclosure relates generally to the field of non-invasive brain stimulation and brain imaging and more specifically, to a wearable headband device for non-invasive brain stimulation and brain imaging with an advanced electronic circuit for significantly improved user experience, multiple electrical stimulation waveforms and safety of users wearing the wearable headband device.

### BACKGROUND

The brain is made up of billions of interconnected neurons and other cell types in connected networks. This complex network of neurons is responsible for processing sensory input, generating motor commands, and controlling various behaviours and cognitive functions. Stimulation technologies that affect electric fields and electrochemical signalling in neurons can modulate the pattern of neural activity and cause altered behaviour, cognitive states, perception, and motor output. Existing brain stimulation devices and systems are mostly designed, developed, and mostly focussed on a hospital use case with large systems setups, which requires operation by a trained specialist. Further, the conventional stimulation devices and systems are very bulky in size and are mostly non-portable units that are difficult to carry due to which such systems are costly and employed only in a limited number of hospitals. Furthermore, with some recent advances some portable devices have been developed, but still are bulky and mostly cover the entire scalp of the brain in the form of a cap like wearable units. However, such systems still require a connection to large analysers to carry out any meaningful analysis of a health state and for non-invasive brain stimulation.

Traditionally, brain stimulation systems include electrical or sensory stimulation, such as electroconvulsive therapy (ECT) are big in size, bulky, complex, costly, which is not desirable. Furthermore, traditional brain stimulation systems that perform testing and determining stimulation points are time-consuming that are found to affect patient cooperation. With conventional wearable devices that are involved in handling of current for stimulation purpose there is further a valid concern of user safety. Therefore, there exists a technical problem of how to develop an advanced and easy-to-use wearable device with advanced electronics that can solve the problem of overcurrent flow and user safety.

Therefore, in light of the foregoing discussion, there exists a need to overcome the aforementioned drawbacks associated with the conventional methods and systems of non-invasive brain stimulation.

### SUMMARY

The present disclosure provides a wearable headband device for non-invasive brain stimulation. The present disclosure provides a solution to the existing problem of how to detect and control the overcurrent flow and other potential safety hazards in the electronic devices that are used for the traditional stimulation methods for brain stimulation. An objective of the present disclosure is to provide a solution that overcomes at least partially the problems encountered in the prior art and provides an improved wearable headband device for non-invasive brain stimulation with minimum components and reduced cost.

One or more objectives of the present disclosure are achieved by the solutions provided in the enclosed independent claims. Advantageous implementations of the present disclosure are further defined in the dependent claims.

In one aspect, the present disclosure provides a wearable headband device for non-invasive brain stimulation, comprising a first stimulation electrode and a second stimulation electrode and an electronic circuit for switching the direction of flow of current between the first stimulation electrode and the second stimulation electrode and also control the amount of current between the first stimulation electrode and second stimulation electrode. The electronic circuit comprises a first circuit that comprises a pair of first type of switches and a pair of second type of switches that are electrically connected to each other, wherein the first circuit is further electrically connected to the first stimulation electrode and the second stimulation electrode. The electronic circuit further comprises a plurality of second circuits configured to control an operation of the pair of first type of switches and the pair of second type of switches of the first circuit. The electronic circuit further comprises a protection circuit that is configured to detect and control the flow of current passing through the first stimulation electrode and the second stimulation electrode via the first circuit, based on a voltage drop across a current sense circuit of the protection circuit. The electronic circuit further comprises a microcontroller that is configured to control the pair of first type of switches and the pair of second type of switches of the first circuit through the plurality of second circuits for the switching the direction of flow of current between the first stimulation electrode and the second stimulation electrode in the non-invasive brain stimulation it also gives instruction for amount of current to be flown between the first stimulation electrode and the second stimulation electrode.

The disclosed wearable headband device is a non-invasive brain stimulation device, which means the wearable headband device does not require any surgical procedures for non-invasive brain stimulation. Beneficially, the development of a new circuit (referred to as a first circuit) that comprises the low-power switches enables to switch the direction of current flow between the first stimulation electrode and the second stimulation electrode. This in turn allowing for a more targeted and effective stimulation with improved accuracy. Further, the plurality of second circuits is designed and developed to accurately control an operation of the pair of first type of switches and the pair of second type of switches of the first circuit, for example, to turn ON or OFF a first type of switch and a second type of switch at the same time (or within a few microseconds) based on command from the microcontroller. This provides a capability to control the switching of the direction of current flow between the first stimulation electrode and the second stimulation electrode and raising errors if anything goes wrong. Furthermore, the wearable headband device includes the protection circuit that is used to prevent overcurrent flow and ensure patient safety at a reduced cost, thereby providing a technical advancement as well as providing the wearable headband device that is economically viable. Advantageously, the invention employs another new circuit specifically developed to ensure safety for users wearing the wearable headband device by automatically detecting and controlling the flow of current passing through the first stimulation electrode and the second stimulation electrode via the first circuit, based on the voltage drop across a current sense circuit of the protection circuit.

It has to be noted that all devices, elements, circuitry, units and means described in the present application could be implemented in the software or hardware elements or any kind of combination thereof. All steps which are performed by the various entities described in the present application as well as the functionalities described to be performed by the various entities are intended to mean that the respective entity is adapted to or configured to perform the respective steps and functionalities. Even if, in the following description of specific embodiments, a specific functionality or step to be performed by external entities is not reflected in the description of a specific detailed element of that entity which performs that specific step or functionality, it should be clear for a skilled person that these methods and functionalities can be implemented in respective software or hardware elements, or any kind of combination thereof. It will be appreciated that features of the present disclosure are susceptible to being combined in various combinations without departing from the scope of the present disclosure as defined by the appended claims.

Additional aspects, advantages, features, and objects of the present disclosure would be made apparent from the drawings and the detailed description of the illustrative implementations construed in conjunction with the appended claims that follow.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1A is a block diagram of a wearable headband device for non-invasive brain stimulation, in accordance with an embodiment of the present disclosure;

FIG. 1B is a block diagram that depicts a first printed circuit board (PCB) of a wearable headband device for non-invasive brain stimulation, in accordance with an embodiment of the present disclosure;

FIG. 2 is a diagram that depicts various exemplary components of a brain stimulation circuit, in accordance with an embodiment of the present disclosure; and

FIGS. 3A and 3B are different diagrams that depict different exemplary scenarios of a wearable headband device for non-invasive brain stimulation to be worn by a user, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

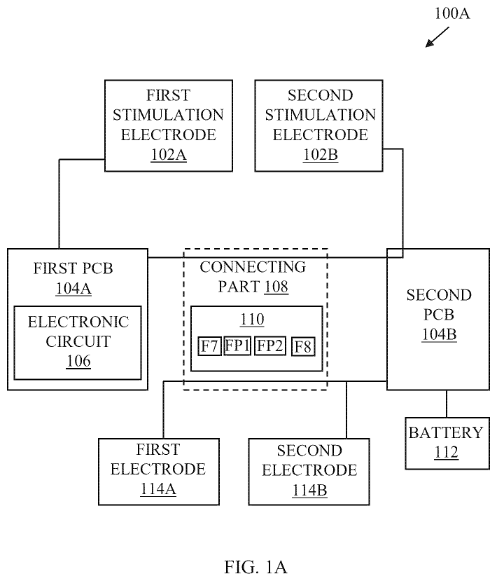

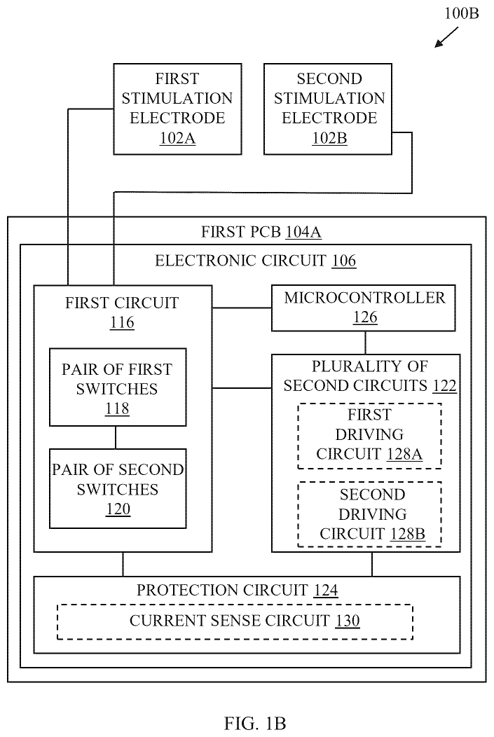

FIG. 1A is a block diagram of a wearable headband device for non-invasive brain stimulation, in accordance with an embodiment of the present disclosure. With reference to the FIG. 1A, there is shown a block diagram of a wearable headband device 100A for non-invasive brain stimulation. The wearable headband device 100A includes a first stimulation electrode 102A and a second stimulation electrode 102B. There is further shown a first printed circuit board (PCB) 104A, a second PCB 104B, and an electronic circuit 106.

The wearable headband device 100A is used for non-invasive brain stimulation, which uses low-intensity electrical currents to stimulate specific regions of the brain. The wearable headband device 100A is typically designed to be worn on the head of a user and can be used to treat a variety of conditions, including but not limited to depression, anxiety, migraine, mild cognitive impairment (MCI), and chronic pain.

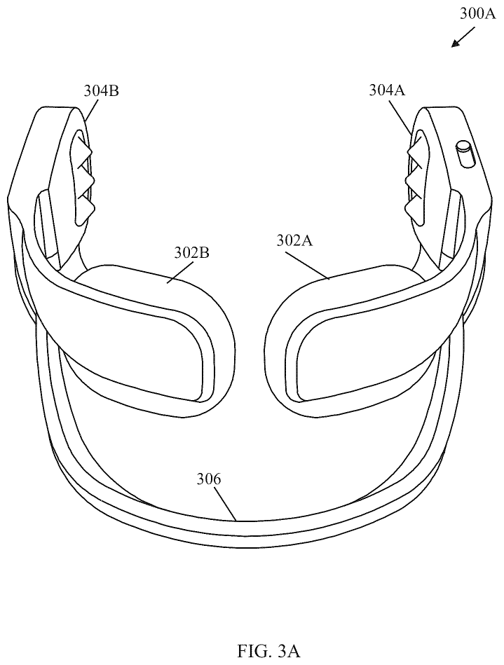

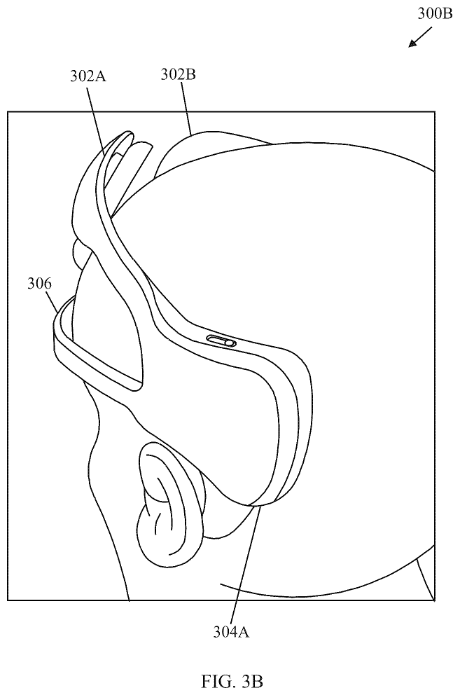

The wearable headband device 100A includes two earpieces, such as a left earpiece and a right earpiece, as further shown and described in FIG. 3A. Moreover, each earpiece includes a separate PCB. For example, the right earpiece includes the first PCB 104A and the left earpiece includes the second PCB 104B. In another example, the right earpiece includes the second PCB 104B and the left earpiece includes the first PCB 104A. In addition, the first PCB 104A is configured to control the first stimulation electrode 102A and the second stimulation electrode 102B. In an example, each of the first stimulation electrode 102A and the second stimulation electrode 102B includes a conductive electrode, a conductive glue, a metal electrode, and a joint. Moreover, the first stimulation electrode 102A is directly connected to the first PCB 104A, and the second stimulation electrode 102B is connected to the first PCB 104A through a connecting part 108. For example, the second stimulation electrode 102B is connected to the first PCB 104A through an electric wire, which passes through the left earpiece and also through the connecting part 108. There is further shown that the first PCB 104A includes the electronic circuit 106, which is configured for switching a direction of flow of current between the first stimulation electrode 102A and the second stimulation electrode 102B, as further shown and described in FIG. 1B. The electronic circuit 106 is used for precise control of current flow to the first stimulation electrode 102A and the second stimulation electrode 102B without causing fluctuations and protects overcurrent flow, ensuring the safety of a patient. In an example, each of the first stimulation electrode 102A and the second stimulation electrode 102B corresponds to a transcranial electrical stimulation (tES) electrode. Moreover, each of the first stimulation electrode 102A, and the second stimulation electrode 102B, uses an electrical current to modulate the activity of specific regions of the brain.

In an implementation, the second PCB 104B includes another circuit that is used for electroencephalography (EEG), which is a non-invasive technique used to measure the electrical activity of the brain via electrodes placed on the scalp, such as through EEG electrode circuit 110 that are arranged in the connecting part 108. There are further shown four numbers of frontal lobes (i.e., F7, FP1, FP2, and F8) that indicate specific locations on a scalp where the EEG electrode circuit 110 is placed. Such specific locations are used for EEG electrode placement to ensure consistent and comparable results. However, the points or the specific locations on the scalp where the EEG electrode circuit 110 is placed may change without affecting the scope of the present disclosure. Moreover, the second PCB 104B is further configured to control a first electrode 114A and a second electrode 114B. In an example, the first electrode 114A is a reference electrode (i.e., SRB1 electrode) and the second electrode 114B a BIAS electrode, which is used in EEG studies. In an implementation, the second PCB 104B is configured to receive current from a battery 112 and transfer the current to the first PCB 104A through the connecting part 108 (e.g., using electric wires). Moreover, the second PCB 104B is also configured to use the battery 112 to supply current to EEG electrode circuit 110, the first electrode 114A, and the second electrode 114B. In an example, the value of the battery 112 ranges from 2 volts (V) to 5 volts. In another example, the value of the battery 112 ranges from 2.8 V to 4.2 V.

The wearable headband device 100A for non-invasive brain stimulation also prevents the overflow of current when providing stimulation. The wearable headband device 100A includes the first stimulation electrode 102A, the second stimulation electrode 102B, and the electronic circuit 106 for switching the direction of flow of current between the first stimulation electrode 102A and the second stimulation electrode 102B. The electronic circuit 106 is configured to track the flow of current with respect to an ideal wave, control current parameters as per Bluetooth command, and notify the user about any error safely. In addition, the electronic circuit 106 is also configured to stop the flow of current during any short circuit or an open circuit. The electronic circuit 106 is used for precise control of current flow to the electrodes without causing fluctuations. Additionally, the electronic circuit 106 is used for accurate control of current flow to different electrodes and protects overcurrent flow, ensuring the safety of the patient.

FIG. 1B is a block diagram that depicts a first printed circuit board (PCB) of a wearable headband device for non-invasive brain stimulation, in accordance with an embodiment of the present disclosure. FIG. 1B is described in conjunction with elements from FIG. 1A. With reference to FIG. 1B, there is shown a block diagram 100B that depicts the first PCB 104A of the wearable headband device 100A for non-invasive brain stimulation. The first PCB 104A includes the electronic circuit 106.

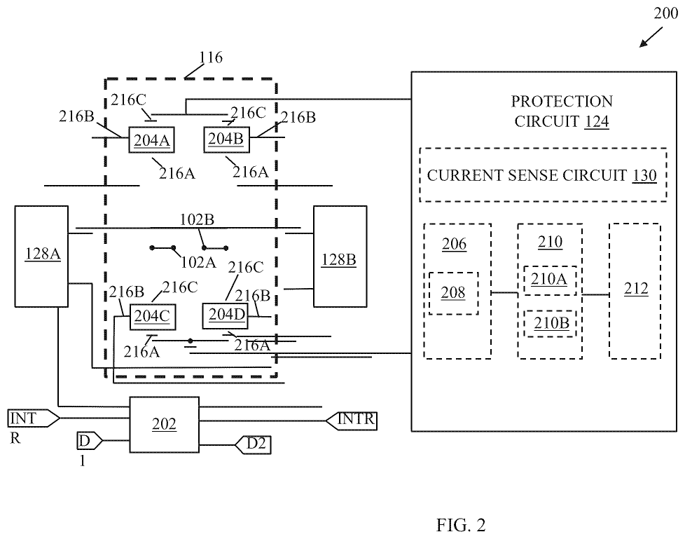

In operation, the electronic circuit 106 is configured for switching a direction of the flow of current between the first stimulation electrode 1020A and the second stimulation electrode 102B. The electronic circuit 106 includes a first circuit 116 that includes a pair of first type of switches 118 and a pair of second type of switches 120 that are electrically connected to each other. Moreover, the first circuit 116 is further electrically connected to the first stimulation electrode 102A and the second stimulation electrode 102B, as shown in FIG. 1B. In an implementation, the first circuit 116 is a modified H-bridge circuit that includes the pair of first type of switches 118 and the pair of second type of switches 120, as further shown and described in FIG. 2. In an implementation, the first circuit 116 is further connected with the battery 112 that acts as a power source and is connected to the second PCB 104B (of FIG. 1A). In such implementation, the first circuit 116 is preferably connected with the battery 112 through a voltage converter. In an example, a voltage of the battery 112 ranges from 2 volts to 5 volts. In another example, the voltage of the battery 112 preferably ranges from 2.8 volts to 4.2 volts. However, the voltage of the battery 112 can vary without limiting the scope of the present disclosure. Moreover, a voltage converter is used to receive voltage from the battery 112 and provide an improved voltage value (e.g., up to 20 volts) to the first PCB 104A and a voltage value of 3v to the first PCB 104A and to the second PCB 104B.

In addition, the first circuit 116 is configured to steer the flow of current from the power source toward the first stimulation electrode 102A and the second stimulation electrode 102B via the pair of first type of switches 118 and the pair of second type of switches 120 when the wearable headband device 100A receives a command from a user device (e.g., from a mobile phone) through the microcontroller 126. Therefore, if the wearable headband device 100A receives the command from the user (e.g., through a user device), then the modified H-bridge circuit is configured to steer the flow of current toward the first stimulation electrode 102A and the second stimulation electrode 102B via the pair of first type of switches 118 and the pair of second type of switches 120.

The electronic circuit 106 further includes a plurality of second circuits 122 that are configured to control an operation of the pair of first type of switches 118 and the pair of second type of switches 120 of the first circuit 116. However, the electronic circuit 106 can also be used in a non-wearable devices or invasive brain simulations without affecting the scope of the present disclosure. In an example, the plurality of second circuits 122 corresponds to H-bridge driver circuits that are configured to control (or drive) the operation of the first circuit 116, such as to control the flow of current through the first stimulation electrode 102A and the second stimulation electrode 102B. The electronic circuit 106 further includes a protection circuit 124 that is configured to detect and control the flow of current passing through the first stimulation electrode 102A and the second stimulation electrode 102B via the first circuit 116, based on a voltage drop across the current sense circuit 130 in the protection circuit 124. The protection circuit 124 is beneficial to ensure that the current flow passing through the first stimulation electrode 102A and the second stimulation electrode 102B remains within safe limits and also ensures the safety of a user by preventing any potential harm from excessive current flow.

The electronic circuit 106 further includes a microcontroller 126 that is configured to control the pair of first type of switches 118 and the pair of second type of switches 120 of the first circuit 116 through the plurality of second circuits 122 for the switching the direction of flow of current between the first stimulation electrode 102A and the second stimulation electrode 102B in the non-invasive brain stimulation. Therefore, the microcontroller 126 is used for precise and controlled switching of the current flow direction between the first stimulation electrode 102A and the second stimulation electrode 102B in the non-invasive brain stimulation. In an implementation, the switching of the direction of the flow of current further includes reversing a polarity or the direction of the flow of current between the first stimulation electrode 102A and the second stimulation electrode 102B. As a result, the electronic circuit 106 allows for improved and specific targeting of the brain stimulation specific areas of the brain, as well as the electronic circuit 106 can vary the stimulation intensity and duration. In addition, the microcontroller 126 can also be programmed to perform specific stimulation protocols, such as alternating between different current flow directions at specific intervals or in response to external triggers, such as a patient's brain activity. Additionally, the microcontroller 126 can communicate with an external device, such as a computer or smartphone, to receive input from the patient or to provide information on the stimulation parameters and effects. As a result, the use of the microcontroller 126 in the electronic circuit 106 provides a high level of precision and control in the non-invasive brain stimulation process. In an implementation, the microcontroller 126 is further configured to detect a fault in the electronic circuit 106 and open the pair of first type of switches 118 and the pair of second type of switches 120 of the first circuit 116 in response to the fault. In an example, the microcontroller 126 is programmed with a set of fault detection algorithms that continuously monitor the electronic circuit 106 for any potential faults. The set of fault detection algorithms are used by the microcontroller 126 to detect abnormal voltage, abnormal current levels, abnormal temperature variations, or other abnormal parameters that could indicate the fault in the electronic circuit 106. Thereafter, the microcontroller 126 is configured to open the pair of first type of switches 118 and the pair of second type of switches 120 to interrupt the flow of current through the first stimulation electrode 102A and the second stimulation electrode 102B, effectively shutting down the electronic circuit 106 until the fault is resolved. In an example, the microcontroller 126 is further configured to shut down the first circuit 116, the plurality of second circuits 122, and the protection circuit 124 to protect the patient. As a result, the microcontroller 126 is beneficial to ensure the safety of the user by preventing any potential harm caused by a malfunctioning condition in the electronic circuit 106 and protecting the user from the overcurrent simulation.

In accordance with an embodiment, the protection circuit 124 is configured to cut off the overcurrent passing to the first stimulation electrode 102A and the second stimulation electrode 102B without any intervention of the microcontroller 126. The overcurrent passed through the electrodes (i.e., the first simulation electrode 102A and the second simulation electrode 102B) could potentially harm the user. Therefore, by automatically cutting off the current without waiting for instructions from the microcontroller 126, the wearable headband device 100A can swiftly prevent any potential risks to the user's well-being.

In an implementation, the switching of the current flow is performed based on demographic data, scores on tests and/or a change in a pattern of electroencephalography (EEG) data and based on one or more transcranial electrical stimulation (tES) application criteria when the wearable headband device 100A is worn by a user. In an example, the tES application criteria further may also include but are not limited to tDCS (transcranial direct current stimulation), tACS (transcranial alternating current stimulation), tRNS (transcranial random noise stimulation), tPCS (transcranial pulsating current stimulation), and the like. Moreover, the wearable headband device 100A for non-invasive brain stimulation may also be referred to as an EEG-controlled transcranial Electrical stimulation (tES) headband. In an example, the wearable headband device 100A includes EEG sensors that are used to measure the electrical activity of the brain, which can be used to detect changes in brain activity patterns. Moreover, the microcontroller 126 is configured to analyze the EEG data in real-time and detect changes in the brain activity patterns, such as based on the detected changes in the brain activity patterns, the microcontroller 126 can adjust the current flow or the direction through the stimulation electrodes. Optionally, the microcontroller 126 is configured to collect the EEG data in real-time and transmit the data to the cloud for processing through the paired computer or smartphone or on the paired computer/phone for detecting changes in the brain activity patterns. Moreover, the switching of the amount of the current flow can be manually set by an operator. As a result, the microcontroller 126 allows the wearable headband device 100A to adapt the stimulation parameters in real-time, providing a more personalized and effective stimulation. In addition, the wearable headband device 100A is also configured to switch the current flow based on the one or more tES application criteria, such as FALL-D parameters (e.g., frequency, amplitude, latency, location), the duration of stimulation (D), duty cycle, interpulse interval, the intensity of the current. The microcontroller 126 is programmed with the one or more tES application criteria and adjusts the current flow accordingly to ensure that the stimulation is delivered according to the one or more tES application criteria, which may be beneficial to allow a more personalized and effective non-invasive brain stimulation.

In accordance with an embodiment, the protection circuit 124 is further configured to provide overvoltage protection between the first stimulation electrode 102A and the second stimulation electrode 102B. The protection circuit 124 is configured to detect the flow of current passing through the first simulation electrode 102A and the second simulation electrode 102B based on the voltage drop across the current sense circuit 130. If an overvoltage condition occurs, where the voltage drop exceeds a predefined threshold, the protection circuit 124 cuts off the overcurrent flowing to the first simulation electrode 102A and the second simulation electrode 102B. As a result, by automatically cutting off the voltage between the first simulation electrode 102A and the second simulation electrode 102B, the wearable headband device 100A can swiftly prevent any potential risks to the user's well-being.

In an implementation, the microcontroller 126 is configured for the switching the direction of the flow of current between the first stimulation electrode 102A and the second stimulation electrode 102B in the non-invasive brain stimulation at a frequency of 10 Hz for a session of a desired time period, such as for 20 minutes (i.e., frequency of switching is 10 times per second). However, the frequency may vary from one user to another user for different sessions. In an example, the frequency ranges from 8 Hz to 16 Hz for 20 minutes. In another example, the frequency ranges from 10 Hz to 20 Hz for 30 minutes, and the like. In an example, a waveform of the frequency for the non-invasive brain stimulation may be a pulsating waveform, a direct current waveform, a variable waveform, and an alternating waveform. Optionally, the pulsating waveform can be a pulsating sine waveform, a pulsating triangular waveform, a pulsating square waveform, and a pulsating complex waveform. Similarly, the alternating waveform can be an alternating sine waveform, an alternating triangular waveform, an alternating square waveform, and an alternating complex waveform. However, other combinations of the waveform for non-invasive brain stimulation can be selected without limiting the scope of the invention.

In an implementation, the plurality of second circuits 122 includes a first driving circuit 128A that is configured to control a switch of the pair of second type of switches 120 and further control another switch of the pair of first type of switches 118 when a first command is received via a first pin of the microcontroller, as further shown and described in FIG. 2. In such implementation, the plurality of second circuits 122 further a second driving circuit 128B that is configured to control yet another switch of the pair of second type of switches 120 and further control another switch of the pair of first type of switches 118 when a second command is received via a second pin of the microcontroller 126, as further shown and described in FIG. 2. In other words, the first driving circuit 128A and the second driving circuit 128B are configured to control the pair of first type of switches 118 and the pair of second type of switches 120 when commands are received via the microcontroller 126, such as by selectively turning on and off the pair of first type of and second type of switches. By controlling the switching of the pair of first type of switches 118 and the pair of second type of switches 120, the plurality of second circuits 122 can control the direction of current flow through the first stimulation electrode 102A and the second stimulation electrode 102B. As a result, the wearable headband device 100A allows for precise control of the current flow through the use of the plurality of second circuits 122 and the microcontroller 126.

In an implementation, the flow of current that passes through the first stimulation electrode 102A and the second stimulation electrode 102B causes a potential difference across a current sense circuit 130 of the protection circuit 124. In an example, the current sense circuit 130 is configured to determine an overcurrent passing through the first stimulation electrode 102A and the second stimulation electrode 102B based on the potential difference caused across the current sense circuit 130 of the protection circuit 124. In such implementation, the protection circuit 124 is further configured to determine if a voltage drop exceeds a pre-defined threshold, and in response to an increased voltage drop, the protection circuit 124 is configured to cut off the overcurrent passing through the first stimulation electrode 102A and the second stimulation electrode 102B. In an example, the protection circuit 124 is configured to monitor the potential difference in real-time and compares the potential difference to a pre-defined threshold (i.e., a safe voltage limit) so as to determine if the voltage drop exceeds the pre-defined threshold or not. Moreover, if the voltage drop exceeds the safe limits, then the protection circuit 124 is configured to immediately interrupt the current flow and trigger an alarm to alert the user. As a result, the protection circuit 124 prevents any potential harm from excessive current flow and ensures the safety of the user. In such implementation, the protection circuit 124 is configured to cut off the overcurrent independent of receipt of any command from the microcontroller 126, as further shown and described in FIG. 2. Beneficially as compared to conventional approaches, the protection circuit 124 is designed to ensure safety automatically and independently for the user. In such implementation, the protection circuit 124 is configured to cut off the overcurrent passing to the first stimulation electrode 102A and the second stimulation electrode 102B without depending on a polarity of the flow of current across the first stimulation electrode 102A and the second stimulation electrode 102B. As a result, the protection circuit 124 provides an added level of security for different users, for example, by safeguarding the patient from overcurrent, as the protection circuit 124 can detect and respond to potential hazards without the need for manual intervention. Furthermore, if the current bypassing circuit 212 in the protection circuit 124 fails to operate, then, in that case, a secondary check is performed by the AND gate 202. Thereafter, the microcontroller 126 is configured to send a stop command to the AND gate 202 and the converter circuit 210 in order to make the protection circuit 124 fail-proof.

In an implementation, the direction of flow of current from the first stimulation electrode 102A to the second stimulation electrode 102B is maintained for a defined time interval based on electroencephalography (EEG) data of the user wearing the wearable headband device 100A. In another implementation, the direction of flow of current from the second stimulation electrode 102B to the first stimulation electrode 102A is maintained for a defined time interval based on the EEG data of the user that is wearing the wearable headband device 100A. Moreover, the time interval can be different in both situations. In other words, the wearable headband device 100A may use demographic data, scores on tests and/or EEG data to determine the direction and duration of the defined time interval for electrical current flow between two stimulation electrodes, which are placed on the head. In an example, the defined time interval can vary for different users based on the EEG data of the corresponding user (e.g., two seconds, five seconds, ten seconds, and the like). In another example, the defined time interval can vary for different users based on the FALL-D and other parameters.

In accordance with an embodiment, the plurality of other defined parameters associated with the flow of current from the first electrode 102A to the second stimulation electrode 102B and vice-versa comprises amplitude, frequency, phase, ramp-up time, ramp-down time, pulse duration, inter-pulse duration, polarity, and time duration of the flow of current. In an implementation, the plurality of other defined parameters associated with the flow of current form the first electrode to the second simulation electrode 102B includes the amplitude and the frequency. In another implementation, the plurality of other defined parameters associated with the flow of current from the first electrode to the second stimulation electrode 102B includes the amplitude, the frequency, the phase, the ramp-up time, and the ramp-down time.

The wearable headband device 100A is a non-invasive brain stimulation device, which means the wearable headband device 100A does not require any surgical procedures for non-invasive brain stimulation. The wearable headband device 100A uses the first circuit 116, such as the modified H-bridge circuit with low-power switches to switch the direction of current flow between the first stimulation electrode 102A and the second stimulation electrode 102B, allowing for a more targeted and effective stimulation with reduced cost. The wearable headband device 100A can be configured to adapt the current flow direction based on changes in the brain activity patterns, allowing for a more personalized and effective stimulation. Moreover, the wearable headband device 100A can be customized (e.g., based on the FALL-D parameters) to the individual patient, making it more comfortable, effective, and easy to use. The wearable headband device 100A further includes the microcontroller 126 for precise current control and the protection circuit 124 to prevent overcurrent flow. The wearable headband device 100A is designed to be worn as a headband, making it portable and convenient for the user providing greater flexibility and convenience.

FIG. 2 is a diagram that depicts various exemplary components of a brain stimulation circuit, in accordance with an embodiment of the present disclosure. FIG. 2 is described in conjunction with elements from FIG. 1A and 1B. With reference to FIG. 2, there is shown a diagram that includes the first circuit 116 and the protection circuit 124. There is further shown the first driving circuit 128A and the second driving circuit 128B are connected with an AND gate 202, which is further connected to the microcontroller 126 (e.g., through pins D1 and D2).

In an implementation, the first circuit 116 is a modified H-bridge circuit that includes a first switch 204A (Q1), a second switch 204B (Q2), a third switch 204C (Q3), and a fourth switch 204D (Q4). Examples of implementation of the first switch 204A, the second switch 204B, the third switch 204C, and the fourth switch 204D may include but are not limited to low-power transistor switches, such as a p-channel metal-oxide-semiconductor (PMOS) switch, a n-channel metal-oxide-semiconductor (NMOS), and the like. There is further shown that the AND gate 202 is configured to receive a first command from a first pin (e.g., through D1) of the microcontroller 126 to control the first driving circuit 128A. In addition, the AND gate 202 is further configured to receive a second command from a second pin (e.g., through D2) of the microcontroller 126 to control the second driving circuit 128B. Moreover, the second driving circuit 128B is configured to control the second switch 204B, and the third switch 204C. In an implementation, a third terminal 216C of the first switch 204A is electrically connected to the second switch 204B through the protection circuit 124, as shown in FIG. 2. Moreover, the second switch 204B is electrically connected to the second driving circuit 128B. Furthermore, the second switch 204B is electrically connected to the fourth switch 204D through the second stimulation electrode 102B. In addition, the third switch 204C is electrically connected to the fourth switch 204D through the protection circuit 124, as shown in FIG. 2.

In an implementation, the third switch 204C is electrically connected to the second driving circuit 128B, and the fourth switch 204D is electrically connected to the first driving circuit 128A. As a result, the first driving circuit 128A is configured to control the first switch 204A of the pair of first type of switches 118 (of FIG. 1B) and further control the fourth switch 204D of the pair of second type of switches 120 (of FIG. 1B) when the first command is received via the first pin of the microcontroller 126. In addition, the second driving circuit 128B is configured to control the second switch 204B of the pair of first type of switches 118 (of FIG. 1B) and further control the third switch 204C of the pair of second type of switches 120 (of FIG. 1B) when the second command is received via the second pin of the microcontroller 126.

In an implementation, the protection circuit 124 includes a Comparator circuit 206, followed by a converter circuit 210, and a current bypass circuit 212. The converter circuit 210 further includes a voltage divider circuit 210A and a voltage buffer 210B. Moreover, the Comparator circuit 206 includes an operational amplifier 208 that is connected to the first circuit 116. In addition, the Comparator circuit 206 of the protection circuit 124 is used to determine an overcurrent passing through the first stimulation electrode 102A and the second stimulation electrode 102B based on the potential difference caused across the current sense circuit 130 in protection circuit 124. Furthermore, in response to an increased potential difference, the protection circuit 124 is configured to cut off the overcurrent passing through the first stimulation electrode 102A and the second stimulation electrode 102B independent of receipt of any command from the microcontroller 126. Moreover, the Comparator Circuit 206 is configured to bypass the current flowing through the first stimulation electrode 102A and the second stimulation electrode 102B by providing an alternative path to the current via the current bypass circuit 212 of the protection circuit 124. In addition, the voltage buffer 210B is configured to develop an interrupt (INTR) signal for the microcontroller 126 to take further action. In an example, the position of the voltage divider circuit 210A and the voltage buffer 210B can be changed without limiting the scope of the present disclosure. Optionally, multiple voltage buffers and multiple voltage divider circuits can be added to improve the efficiency of the protection circuit and have more protection parameters to protect the patients from the extra flow of current.

The voltage divider circuit 210A is configured to generate INTR signal that is used to prevent the overflow of current when providing stimulation and also ensures the safety of a user by preventing any potential harm from excessive current flow. As a result, the protection circuit 124 is beneficial to ensure the safety of the user by preventing any potential harm, which may be caused by overcurrent passing through the first stimulation electrode 102A and the second stimulation electrode 102B. Beneficially as compared to the conventional approach, the Comparator circuit 206 is configured to measure the potential difference across the current sense circuit 130 in the protection circuit 124. In addition, the protection circuit 124 further provides an interrupt signal to the AND gate 202. As a result, the AND gate 202 can interrupt the operation of the first stimulation electrode 102A and the second stimulation electrode 102B without interruption of microcontroller 126. The AND gate 202 is an added protection to provide overcurrent-protection in a case where bypass circuit 212 fails to work. The operational amplifier 208 is also connected to a microcontroller pin for microcontroller 126 to continuously monitor voltage as a second-check in over-current protection between the first stimulation electrode 102A and the second stimulation electrode 102B. The microcontroller 126 further measures voltage and transmit a signal to bypass and shut down the stimulation circuit in case the current overflows.

FIGS. 3A and 3B are different diagrams that depict different exemplary scenarios of a wearable headband device for non-invasive brain stimulation to be worn by a user, in accordance with an embodiment of the present disclosure. FIGS. 3A and 3B are described in conjunction with elements from FIGS. 1A, 1B, and 2. With reference to the FIG. 3A, there is shown a diagram 300A that depicts an exemplary scenario of the wearable headband device 100A for non-invasive brain stimulation (i.e., a front view). With reference to the FIG. 3B, there is shown a diagram 300B that depicts an exemplary scenario of the wearable headband device 100A for non-invasive brain stimulation worn by a user.

The wearable headband device 100A includes a first-end 302A, a second-end 302B, a third-end 304A, a fourth-end 304B, and a fifth-end 306. The first-end 302A and the third-end 304A are arranged on the left side of the wearable headband device 100A. Moreover, the second-end 302B and the fourth-end 304B are arranged on the right side of the wearable headband device 100A. In an example, the first-end 302A is configured to include the first stimulation electrode 102A, and the second-end 302B is configured to include the second stimulation electrode 102B. In another example, the first-end 302A is configured to include the second stimulation electrode 102B, and the second-end 302B is configured to include the first stimulation electrode 102A. In addition, the third-end 304A is configured to enclose or wrap around the second PCB 104B, and the fourth-end 304B is configured to enclose the first PCB 104A. In an example, the third-end 304A is configured to enclose or wrap around the first PCB 104A, and the fourth-end 304B is configured to enclose the second PCB 104B. In another example, the third-end 304A is configured to enclose or wrap around the second PCB 104B as well as to cover the first PCB 104A. In yet another example, the fourth-end 304B is configured to cover the first PCB 104A as well as the second PCB 104B. Moreover, the fifth-end 306 (e.g., a strap) is configured to cover the connecting part 108 as well as house the EEG circuit 110. As a result, the fifth-end 306 is used to secure the connecting part 108 and includes the EEG circuit 110 and holds them in place on the patient's head. The wearable headband device 100A is typically designed to be worn on the head of a user, as shown in FIG. 3B. The wearable headband device 100A is used for non-invasive brain stimulation, which uses low-intensity electrical currents to stimulate specific regions of the brain and can be used to treat a variety of conditions, including depression, anxiety, MCI, migraine, and the like.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural. The word “exemplary” is used herein to mean “serving as an example, instance or illustration”. Any embodiment described as “exemplary” is not necessarily to be construed as preferred or advantageous over other embodiments or to exclude the incorporation of features from other embodiments. The word “optionally” is used herein to mean “is provided in some embodiments and not provided in other embodiments”. It is appreciated that certain features of the present disclosure, which are, for clarity, described in the context of separate embodiments, may also be provided in combination in a single embodiment. Conversely, various features of the invention, which are, for brevity, described in the context of a single embodiment, may also be provided separately or in any suitable combination or as suitable in any other described embodiment of the disclosure.

1. A wearable headband device for non-invasive brain stimulation, comprising:
a first stimulation electrode and a second stimulation electrode; and
an electronic circuit for switching a direction of flow of current between the first stimulation electrode and the second stimulation electrode, wherein the electronic circuit comprises:
a first circuit that comprises a pair of first type of switches and a pair of second type of switches that are electrically connected to each other, wherein the first circuit is further electrically connected to the first stimulation electrode and the second stimulation electrode;
a plurality of second circuits configured to control an operation of the pair of first type of switches and the pair of second type of switches of the first circuit;
a protection circuit that is configured to detect and control the flow of current passing through the first stimulation electrode and the second stimulation electrode via the first circuit, based on a voltage drop across a current sense circuit of the protection circuit; and
a microcontroller that is configured to control the pair of first type of switches and the pair of second type of switches of the first circuit through the plurality of second circuits for the switching the direction of flow of current between the first stimulation electrode and the second stimulation electrode in the non-invasive brain stimulation.

2. The wearable headband device of claim 1, wherein the plurality of second circuits comprises a first driving circuit that is configured to control a first switch of the pair of first type of switches and further control a fourth switch of the pair of second type of switches when a first command is received via a first pin of the microcontroller.

3. The wearable headband device of claim 1, wherein the plurality of second circuits comprises a second driving circuit configured to control a second switch of the pair of first type of switches and further control a fourth switch of the pair of second type of switches when a second command is received via a second pin of the microcontroller.

4. The wearable headband device of claim 1, wherein the first circuit is a modified H-bridge circuit.

5. The wearable headband device of claim 1, wherein in the first circuit, a first terminal of the first switch is electrically connected to a third terminal of a third switch through the first stimulation electrode, a second terminal of the first switch is electrically connected to the first driving circuit, and a third terminal of the first switch is electrically connected to a third terminal of the second switch through the protection circuit.

6. The wearable headband device of claim 5, wherein in the first circuit, a first terminal of the second switch is electrically connected to a third terminal of the fourth switch through the second stimulation electrode, and the second terminal of the second switch is electrically connected to the second driving circuit.

7. The wearable headband device of claim 6, wherein in the first circuit, a first terminal of the third switch is electrically connected to a first terminal of the fourth switch through the protection circuit, a second terminal of the third switch is electrically connected to the second driving circuit, and a second terminal of the fourth switch is electrically connected to the first driving circuit.

8. The wearable headband device of claim 1, the first circuit further connected with a battery that acts as a power source, and wherein the first circuit is configured to steer the flow of current from the power source toward the first stimulation electrode and the second stimulation electrode via the pair of first type of switches and the pair of second type of switches when the wearable headband device receives a command from a user through the microcontroller.

9. The wearable headband device of claim 8, wherein the flow of current that passes through the first stimulation electrode and the second stimulation electrode causes a potential difference across the current sense circuit of the protection circuit.

10. The wearable headband device of claim 9, wherein the current sense circuit that is configured to determine an overcurrent passing through the first stimulation electrode and the second stimulation electrode based on the potential difference caused across the current sense circuit of the protection circuit.

11. The wearable headband device of claim 10, wherein the protection circuit is further configured to determine if a voltage drop across the current sense circuit of protection circuit exceeds a pre-defined threshold, and in response to an increased voltage drop, the protection circuit is configured to cut off the overcurrent passing through the first stimulation electrode and the second stimulation electrode.

12. The wearable headband device of claim 11, wherein if the voltage drop across the current sense circuit of protection circuit exceeds a pre-defined threshold, then the protection circuit is configured to cut off the overcurrent passing to the first stimulation electrode and the second stimulation electrode independent of receipt of any command from the microcontroller.

13. The wearable headband device of claim 11, wherein the protection circuit is configured to cut off the overcurrent passing to the first stimulation electrode and the second stimulation electrode without depending on a polarity of the flow of current across the first stimulation electrode and the second stimulation electrode.

14. The wearable headband device of claim 11, wherein the protection circuit comprises a Comparator circuit and a converter circuit that further includes a voltage divider circuit and a voltage buffer.

15. The wearable headband device of claim 14, wherein the protection circuit is further configured to cut off the overcurrent passing through the first stimulation electrode and the second stimulation electrode, and the comparator circuit is configured to bypass the current flowing through the first stimulation electrode and the second stimulation electrode by providing an alternative path to the current via current bypass circuit of the protection circuit.

16. The wearable headband device of claim 1, wherein the protection circuit is further configured to provide overvoltage protection between the first stimulation electrode and the second stimulation electrode.

17. The wearable headband device of claim 1, wherein the switching of the direction of flow of current further comprises reversing a polarity or the direction of the flow of current between the first stimulation electrode and the second stimulation electrode or changing at least one parameter of a plurality of other defined parameters associated with the flow of current.

18. The wearable headband device of claim 17, wherein the direction and the plurality of other defined parameters associated with the flow of current from the first stimulation electrode to the second stimulation electrode is maintained for a defined time interval based on demographic data, scores on tests and electroencephalography (EEG) data of a user wearing the wearable headband device, wherein the plurality of other defined parameters associated with the flow of current from the first electrode to the second stimulation electrode comprises amplitude, frequency, phase, ramp-up time, ramp-down time, pulse duration, inter-pulse duration, polarity, and time duration of the flow of current.

19. The wearable headband device of claim 1, wherein the microcontroller is further configured to detect a fault in the electronic circuit and as a second check on the protection circuit and in response open the pair of first type of switches and the pair of second type of switches of the first circuit in response to the fault.

20. The wearable headband device of claim 1, wherein the switching an amount of the current flow is performed based on demographic data, scores on tests or a change in a pattern of electroencephalography (EEG) data and based on one or more transcranial electrical stimulation (tES) application criteria when wearable headband device is worn by a user.
